(19) United States
(12) Patent Application Publication
(10) Pub. No.: US 2022/0323247 A1
(43) Pub. Date: Oct. 13, 2022

(54) SOFT ACTIVE HAND ORTHOSIS

(71) Applicant: Aalborg Universitet, Aalborg Øst (DK)

(72) Inventors: Mohamed Abdelhafiz, Aalborg (DK); Lotte Najanguaq Søvsø Adreasen Struijk, Terndrup (DK); Strahinja Dosen, Aalborg (DK); Erika Geraldina Spaich, Klarup (DK)

(73) Assignee: Aalborg Universitet, Aalborg Øst (DK)

(21) Appl. No.: 17/620,337

(22) PCT Filed: Jun. 23, 2020
(86) PCT No.: PCT/DK2020/050185

§ 371 Date: Dec. 17, 2021

(30) Foreign Application Priority Data
Jun. 23, 2019 (DK) PA 2019 00761

(51) Int. Cl. A61F 5/01

(52) U.S. Cl. CPC A61F 5/013; A61F 2005/0139; A61F 2005/0155

(57) ABSTRACT

The invention relates to a hand orthosis (1) for assisting the movements of a user's hand. The hand orthosis comprises a soft glove (2) adapted to receive at least a part of the user's hand with finger sections of the glove receiving each of the user's fingers. It further comprises at least one actuator box (3), and a power transmission (4) for transferring forces from the at least one actuator box to different parts of the glove via exo-tendons (5). The exo-tendons are arranged on a dorsal side, on a palmar side, or on both a dorsal side and a palmar side of the glove. The exo-tendons are arranged so that the glove can assist flexion, extension, or both extension and flexion of the fingers, respectively and so that the exo-tendons act on the fingers to produce motion that mimics the movement of a human hand. The hand orthosis provides different grasp types, self- adaptation of the fingers' motion to the shape of the object, and physiologically appropriate distribution of finger forces.

### FIELD OF THE INVENTION

The present invention relates to a hand orthosis. In particular, it relates to a hand orthosis comprising a soft glove and exo-tendons which are arranged to act on the fingers of a user to produce motion that mimics the movement of a human hand.

### BACKGROUND OF THE INVENTION

A hand orthosis can find use in a number of applications, e.g. where the user needs assistance of the movements of the hand. Such a hand orthosis can also be referred to as an active glove or an exoskeleton. For known hand orthoses, the assistance of the movements can be driven by an actuator connected to the finger regions of the glove via rigid links or tendons.

The main challenges that active gloves, which are actuated by tendons, face are the ability to perform extension and flexion using different types of grasping by the aid of one motor. It is another challenge to make the five fingers of the hand self-adapted to differently shaped objects for all types of grasping. Besides, the movement of the fingers during flexion and extension should be comfortable, in other words, they should respect the biomechanical constraints of the finger during both flexion and extension.

BiomHED is a glove that assists the hand in performing dexterous flexion movements by using three tendons mimicking the human finger tendons. However, it requires more than one actuator for each finger to be able to perform the movements. If the same strategy is used then two extra motors will be required for extension. The need for such additional motors adds weight to the glove and this may make it uncomfortable for the user.

Robo-glove, invented by General Motors, also used tendons, but the system was under-actuated to use one tendon for the flexion of each finger. However, a separate motor was required for each finger to be able to adapt the fingers to irregularly shaped objects, which does not guarantee that the forces are distributed between the fingers physiologically, all the time. In addition, this design increases the weight of the system.

The SEM glove is also a tendon-driven device that flexes the thumb, middle and ring fingers using tendons providing assistive forces during grasping. A pulley system is used to generate forces over the three fingers, but it does not provide extension of the fingers, which is an essential movement particularly for individuals with neurological injuries or diseases in whom the hand is impaired.

To conclude, designing a glove comprising all the above-mentioned features, i.e., physiological flexion and extension movements and finger forces as well as different types of grasps with the fingers shaping adaptively around an object, is still a challenge.

### OBJECT OF THE INVENTION

It is an object of the present invention to provide a hand orthosis with flexion and extension motions that are close to the normal human motions and that is thereby comfortable for the user.

It is another object of the present invention to provide a hand orthosis with which the generated motions comply with the biomechanical constraints of the fingers due to the musculoskeletal and tendon structure.

It is another object of the present invention to provide a hand orthosis allowing for different types of grasping, which are useful for performing activities of daily living. During all types of grasping, the five fingers adapt to objects with different shapes without knowing the shapes and sizes a priori.

It is an object of at least some embodiments of the present invention to provide a hand orthosis in the form of a soft glove that actively flexes and extends the five fingers of the user's hand using one motor and exo-tendons.

It is a further object of the present invention to provide an alternative to the prior art.

In particular, it may be seen as an object of the present invention to provide a hand orthosis that solves the above-mentioned problems of the prior art.

### SUMMARY OF THE INVENTION

The present invention is an active soft hand orthosis that can be used to assist individuals suffering from upper limb impairment due to neuromuscular injuries. It can support performing activities of daily life such as eating, drinking, grasping objects, writing using pens, typing on keypads, and performing other activities related to self-hygiene.

The invention can also be used to assist task-oriented rehabilitation programs to regain the functional movements of the hand after neuromuscular injuries. In these programs, the goal is to train to perform those functional movements, which might be very difficult for the impaired individuals. The invention allows training by supporting the production of the hand's movements. The invention is portable so that it can be used in clinics and in home settings, allowing the user to train extensively to increase the rehabilitation efficiency.

It can be used as a useful tool for providing virtual force resistance in virtual-reality applications. Some virtual-reality applications require applying resistive forces over the fingers when training hand skills in different scenarios like sewing, and training of musicians and surgeons. Likewise, the glove can be used to provide haptic feedback by producing interaction forces between the user and the virtual objects.

Furthermore, it can be used to provide workers, who work with heavy equipment, with powerful hand grasping to the objects they hold. This is useful to delay the development of muscle fatigue and/or avoid work related muscle injuries. This is also useful for elderly people, with weak hands, to strengthen their grasping force.

Thus, the above-described object and several other objects are intended to be obtained in a first aspect of the invention by providing a hand orthosis for assisting the movements of a user's hand, the hand orthosis comprising:
a soft glove adapted to receive at least a part of the user's hand with finger sections of the glove receiving each of the user's fingers,
at least one actuator box, and
a power transmission for transferring forces from the at least one actuator box to different parts of the glove via exo-tendons,

wherein the exo-tendons are arranged on a dorsal side, on a palmar side, or on both a dorsal side and a palmar side of the glove, and wherein the exo-tendons are arranged so that the glove can assist extension, flexion, or both extension and flexion of the fingers, respectively, and so that the exo-tendons act on the fingers to produce motion that mimics the movement of a human hand.

This is achieved by preserving the natural kinematic coupling between the interphalangeal joints during extension and by providing a system of forces across the finger sections that resembles the forces provided by the human flexor tendons during flexion. Furthermore, the exo-tendons also act as a differential mechanism, allowing the finger sections to take the shape of an object to be grasped when getting in contact with the object, resulting in better grasp.

What is called “soft glove” in this description could also be referred to as “glove”. By “soft” is meant that it is so flexible that the glove itself does not add any significant resistance against the movement of the hand.

By “exo-tendon” is meant a flexible cord used to transfer power. An advantage of using such flexible exo-tendons to provide the assisted motion of a user's hand compared to an orthosis based on rigid links is that it is significantly more comfortable for the user. The exo-tendons can e.g. be made from Fluorocarbon or monofilament nylon.

The glove is considered as the part that is directly in contact with the user's hand. It is preferably made from a soft fabric that is used as a base to insert the exo-tendons transmitting the power from the actuator box to the fingers sections of the glove. The exo-tendons are preferably inserted on all the five fingers from both sides, the dorsal (for extension) and the palmar (for flexion). The fact that the glove is typically made from flexible fabric means that it can follow the movements of the hand in a manner that is comfortable to the user. The fabric should preferably be made from a material which is non-irritating to the skin and which is moisture-permeable. Alternatively, the glove can be made from other materials fulfilling the purpose of the hand orthosis, such as a flexible rubber material or leather.

The actuator box is responsible for the movements of the fingers into extension or flexion including all grasping types (e.g. pinch grasping, tripod grasping, power grasping, etc.). In some embodiments of the invention, the actuator box distributes the forces physiologically to the five fingers (i.e., the force on the thumb is always equal to the sum of the forces exerted by the other four fingers). In addition, the finger angles are adaptive to be able to fully embrace objects with different shapes without a priori knowledge of the shape and/or dimension. This feature is also valid for all grasping types.

In embodiments wherein the exo-tendons are arranged on both a dorsal side and a palmar side of the glove, the hand orthosis may be controllable so that only exo-tendons on one side are active at a time.

The hand orthosis should preferably be designed so that the generated motions comply with the biomechanical constraints of the fingers due to the musculoskeletal and tendon structure. Hereby it is meant that it complies with the physiologically plausible angles between the joints of the fingers as well as the natural kinematic coupling between these joints.

In presently preferred embodiments of the invention, the at least one actuator box comprises at least one motor for providing power to the power transmission. However, at least for some applications of the hand orthosis, the power may also be provided from an external device to which the hand orthosis is connected. The motor may e.g. be a DC motor.

The at least one actuator box may comprise exactly one motor. This reduces the overall weight of the device. It, also, reduces the complexity of the controller of the orthosis to grasp irregularly shaped objects.

Furthermore, the at least one actuator box may comprise at least one mini-low power solenoid to control a finger section individually by blocking/unblocking its movement. By “mini-low” is meant a solenoid that generates an amount of force that is enough to move its rod around 5 mm to block the movements.

In any of the above-described embodiments, the exo-tendons may be arranged and controlled so that the forces from the at least one actuator box are distributed equally between a thumb on one side and the rest of the fingers on the other side. Hereby it will be possible to allow the user's fingers to adapt to any irregular object while preserving the equal distribution of power across the two groups of fingers. This equality allows the glove to provide a stable and natural grasp of any irregular object. By “natural grasp” is meant a movement that resembles that of a normally functioning hand not wearing the hand orthosis.

In presently preferred embodiments of the invention, the exo-tendons are arranged and controlled so that the finger sections of the glove can be individually activated. This allows the user wearing the glove to perform different gestures that might otherwise not be possible. This can be obtained by using a differential mechanism where all three phalanges of the finger move together following the normal human flexion.

In any of the embodiments wherein the hand orthosis comprises at least one motor, the exo-tendons may be arranged and controlled so that motor torque is distributed equally between the thumb on one side and the rest of the fingers on the other side to provide the user with a natural grasp. This distribution is typically provided by using an actuator box comprising at least one differential mechanism either using pulleys, differential gears, or springs and pulleys to distribute the motor power between the finger sections.

The exo-tendons may be attached to the glove at the radial and ulnar side of the proximal, middle, and distal phalanges of each finger section. If necessary, further fastening points may be at other positions, such as on the tip of the distal phalanx and on the dorsal side of the middle phalanx.

At least one of the exo-tendons may pass through a Bowden cable along at least a part of its length. Hereby the exo-tendon can be protected, especially at points where it changes direction.

The exo-tendons may pass through Bowden cables from the at least one actuator box to the region of the glove adapted to be located adjacent to the metacarpal bones of the user during use of the hand orthosis.

Thus, in at least some embodiments of the invention, the power transmission system that is used to transfer the power from the actuator box to the glove consists of flexible Bowden cables and soft exo-tendons that run through the Bowden cables. The Bowden cables are fixed at the actuator box from one side and to the user wrist on the other side but they do not stop there. They extend until the end of the metacarpal bones. A Velcro band wrapped around the wrist fixes the Bowden cables at the wrist. The Bowden cables pass through the Velcro and it is fixed from moving by two stoppers attached on the Bowden cables from both sides of the Velcro.

In any of the embodiments as described above, the exo-tendons may be fastened to the glove by use of guidance pearls attached to an outer surface of the glove. If the glove is made from fabric, the guidance pearls could be incorporated into the fabric forming the glove. By “guidance pearls” is preferably meant small bead-like elements having a through-going hole through which the exo-tendons can be led and held in the desired position with respect to the glove while still allowing for the necessary movements. Alternatively or in combination therewith, the exo-tendons can be held in place by other holding means, such as small rings or loops.

In embodiments having such guidance pearls, the exo-tendons may be routed as follows around the corresponding finger section to provide a force system over the finger section that resembles the force system provided by human flexion tendons:
the exo-tendon has a starting point at the end of the first metacarpal bone before reaching the metacarpophalangeal joint, one end of the exo-tendon passes through the guidance pearls on the ulnar side of the finger, ascending upwards until the middle phalanx, surrounding the distal part of the phalanx on the dorsal side, and then descending on the radial side of the finger to be fixed further down beyond the starting point;
the other end of the exo-tendon passes through the guidance pearls on the radial side of the finger, ascending until the distal part of the distal phalanx to descend through the guidance pearls on the ulnar side of the finger, to be fixed at the proximal aspect of the middle phalanx; and
a driver exo-tendon is connected to the exo-tendon at the starting point to be pulled using the at least one actuator box.

By “starting point” is meant a virtual point, from where the definition of the routing of the exo-tendon in relation to the finger was started. This will also be illustrated in the figures.

This flexion mechanism as just described distributes the flexion forces equally over the distal and proximal phalanges throughout the flexion motion (until full flexion), thereby mimicking the human musculoskeletal flexion system. This allows the mechanism to respect the biomechanical constraints between the distal interphalangeal joint (DIP) and the proximal interphalangeal joint (PIP) due to the musculoskeletal and tendon structure, where the PIP angle should be always >1.5 times the DIP angle to result on a comfortable flexion.

Alternatively, each of the exo-tendons may be routed as follows to provide the force system for the corresponding finger section that resembles the force system provided by human flexion tendons:
the exo-tendon has a starting point at the end of the first metacarpal bone before reaching the metacarpophalangeal joint, one end of the exo-tendon passes through the guidance pearls on the ulnar side of the finger, ascending upwards until the middle phalanx, surrounding the distal part of the phalanx on the dorsal side, and then descending on the radial side of the finger;
the other end of the exo-tendon passes through the guidance pearls on the radial side of the finger, ascending until the distal part of the distal phalanx to descend through the guidance pearls on the ulnar side of the finger; and
both ends of the exo-tendons are pulled directly by the at least one actuator box to flex the finger section.

Studies leading to the present invention have shown that by using such routings of the exo-tendons, it is possible to replicate physiological appropriate finger flexion.

Preferably, on the distal phalanx, the exo-tendon passes through a Bowden cable to prevent it from being overtightened around the distal phalanx when the exo-tendons are tightened.

In embodiments comprising guidance pearls, the exo-tendons on the dorsal side of the finger sections of the glove may be arranged so that two exo-tendons are used to extend each finger; the two exo-tendons being arranged so that:
the first exo-tendon passes through a separate piece of Bowden cable on the radial and ulnar side of the metacarpal joint, then through the guidance pearls on the radial and ulnar side of the finger, until the middle phalanx, where the exo-tendon passes through the guidance pearls on the dorsal side of the middle and distal phalanges;
then the first exo-tendon is fixed on the distal phalanx by routing it around the phalanx;
the second exo-tendon passes through a separated piece of Bowden cable on the radial and ulnar side of the metacarpal joint, then through the guidance pearls on the radial and ulnar side of the finger, until the proximal aspect of the proximal phalanx, where the second exo-tendon passes through the guidance pearls on the dorsal side of the proximal and middle phalanges;
then the second exo-tendon is fixed on the middle phalanx by routing it around the phalanx.

Alternatively, each of the exo-tendons may be routed as follows to extend the corresponding finger section, using two exo-tendons:
the first exo-tendon passes through a separate piece of Bowden cable on the radial and ulnar side of the metacarpal joint, then through the guidance pearls on the radial and ulnar side of the finger, until the distal aspect of the proximal phalanx, where the exo-tendon passes through the guidance pearls on the distal aspect of the middle and distal phalanges on the dorsal side of the finger;
then the first exo-tendon is fixed on the distal phalanx by routing it around the phalanx;
the second exo-tendon passes through a separated piece of Bowden cable on the dorsal side of the metacarpal joint, then through the guidance pearls on the dorsal side of the finger, the proximal phalanx, until the distal aspect of the middle phalanges;
then the second exo-tendon is fixed on the middle phalanx by routing it around the phalanx.

Hereby it is found possible to replicate physiologically appropriate finger extension and prevent hyper-extending the distal phalanx.

In any of the above-described embodiments of the invention, the at least one actuator box may comprise at least one motor and a plurality of pulleys used to guide the exo-tendons, wherein the pulleys are adapted and arranged to enable a self-adaptive distribution of the forces from the at least one motor to each of the finger sections of the glove via the power transmission. By “self-adaptive” is meant that the pulley mechanism always adapts itself to distribute the forces equally across the exo-tendons that are responsible to flex the index, middle, ring and little fingers, while the force exerted on the exo-tendon that is responsible to flex the thumb is always equal to the sum of the forces exerted by the other four fingers.

Alternatively, the at least one actuator box may comprise at least one motor and a plurality of differential gears for providing a self-adapted distribution of the forces from the at least one motor to each of the finger sections of the glove via the power transmission.

In yet other alternatives, the at least one actuator box may comprise at least one motor, a plurality of springs and pulleys adapted and arranged to provide a self-adapted distribution of the forces from the at least one motor to each of the finger sections of the glove via the power transmission.

In presently preferred embodiments of the invention, the device is controlled intuitively, by the user, using EMG electrodes. It allows the device to detect the flexor and extensor muscles activities, which consequently could be used to estimate the intention of the user to perform different gestures, using machine-learning techniques. According to that, the device determines the required gesture and performs it automatically. Alternatively, other interfaces can be used, e.g. sensors on the fingers to detect contact or bend sensors to detect slight flexion of the fingers.

In a second aspect, the invention relates to the use of a hand orthosis in any of the above-described embodiments for assisting a task oriented rehabilitation program. Thus, in such a use, the hand orthosis according to the first aspect of the invention is used for assisting opening and closing of the user's hand.

Alternatively, the second aspect of the invention relates to the use of a hand orthosis as described above as a tool for providing virtual force resistance and haptic feedback in virtual-reality applications.

In yet another alternative, the second aspect of the invention relates to the use of a hand orthosis as described above to provide permanent assistance during daily life activities to chronically impaired patients.

In yet another alternative, the second aspect of the invention relates to the use of a hand orthosis as described above to provide workers who work with heavy equipment with powerful hand grasping of the objects they hold. This is useful to delay the development of muscle fatigue and/or avoid work related muscle injuries. This is also useful for elderly people, with weak hands, to strengthen their grasping force.

The first and second aspects of the present invention may be combined. These and other aspects of the invention will be apparent from and elucidated with reference to the embodiments described hereinafter.

### BRIEF DESCRIPTION OF THE FIGURES

The hand orthosis according to the invention will now be described in more detail with regard to the accompanying figures. The figures show some ways of implementing the present invention and is not to be construed as being limiting to other possible embodiments falling within the scope of the attached claim set.

FIG. 1.a schematically shows the overall concept of the invention.

FIG. 1.b shows the relation between the DIP and PIP joint angle trajectories for both voluntary finger flexion and soft hand orthosis flexion to demonstrate the ability of the device to flex the DIP and PIP joints naturally.

FIG. 2 shows the flexion profile of the finger joints for both voluntary flexion and soft hand orthosis flexion to demonstrate the ability of the device to flex the three joints of the finger, MCP, PIP and DIP, naturally.

FIG. 3 represents the relation between the DIP and PIP joint angle trajectories for both voluntary finger extension and soft hand orthosis extension to demonstrate the ability of the device to extend the finger naturally.

FIG. 4 represents the DIP joint angle trajectory for both voluntary finger extension and soft hand orthosis extension to demonstrate the ability of the device to extend the finger without hyper-extending the distal phalanx.

FIG. 5 represents the index and middle fingers when the middle finger is stopped, during finger flexion, to demonstrate the ability of the device to perform different grasping types.

FIGS. 6 and 7 schematically show how the invention can be used to replicate a physiologically appropriate finger extension without suffering from hyperextension of the distal phalanx.

FIGS. 8 and 9 schematically show how the invention can be used to replicate a physiologically appropriate finger flexion.

FIGS. 10-13 schematically show four different designs of an actuator box that can be used for a hand orthosis according to the present invention.

FIG. 14 schematically shows an anti-derailment pulley mechanism. It is used by the actuator boxes shown in FIGS. 11 and 12, where the differential mechanism is developed by a set of differential gears. It assists the exo-tendons, that transmit the power form the actuator to the glove, to be always routed tightly around the shaft of the gearbox and avoid any derailment.

### DETAILED DESCRIPTION OF AN EMBODIMENT

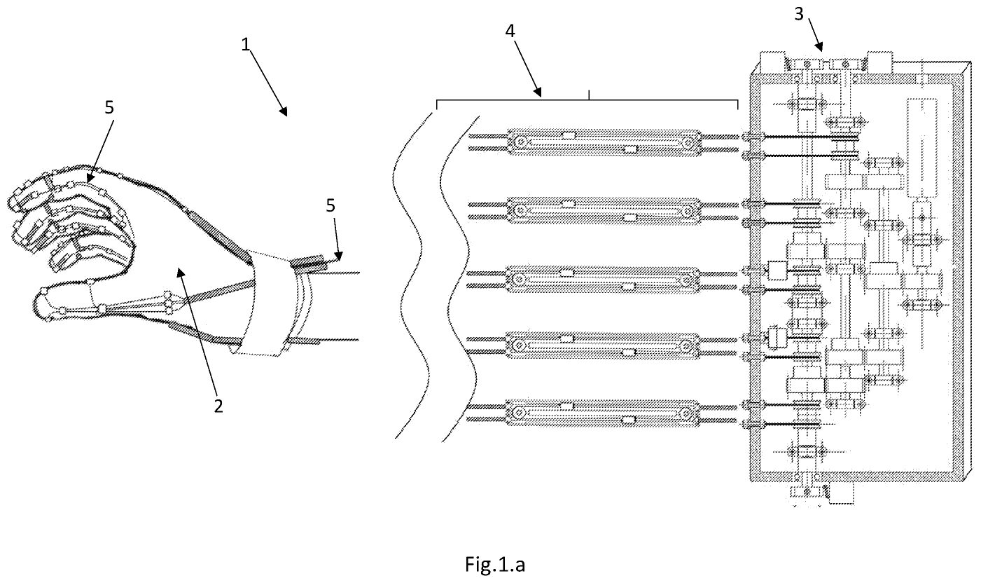

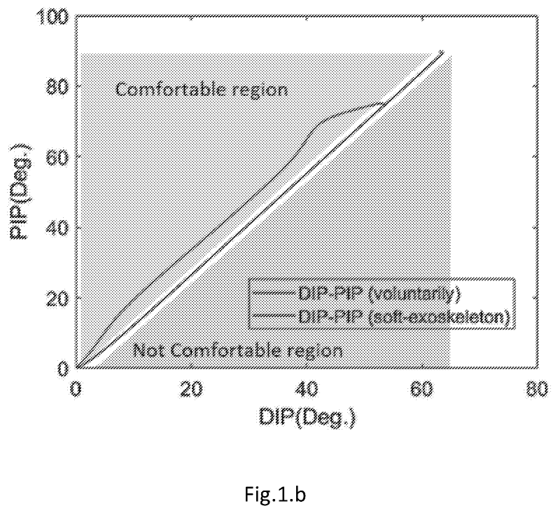

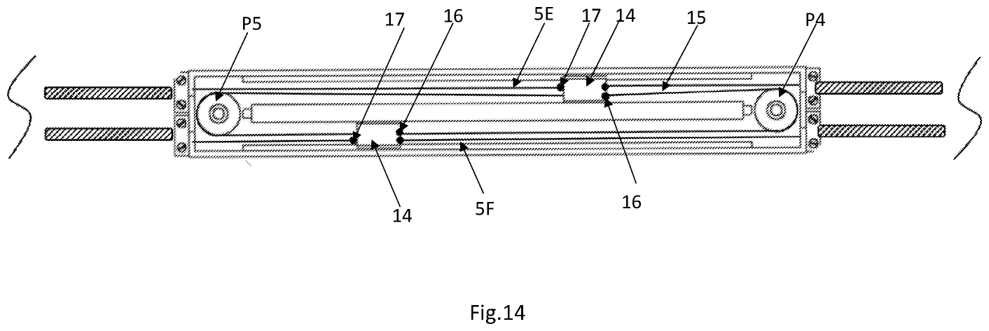

FIG. 1.a schematically shows the overall design of a hand orthosis 1 for assisting the movements of a user's hand. The hand orthosis comprises a soft glove 2 adapted to receive at least a part of the user's hand with finger sections of the glove 2 receiving each of the user's fingers. The hand orthosis 1 in FIG. 1.a further comprises an actuator box 3, and a power transmission 4 for transferring forces from the actuator box 3 to different parts of the glove 2 via exo-tendons 5. In the embodiment as shown in FIG. 1.a, the exo-tendons 5 are arranged on both a dorsal side and a palmar side of the glove 2. They are arranged so that the glove 2 can assist both extension and flexion of the fingers and so that the exo-tendons 5 act on the fingers to produce motion that mimics the movement of a human hand. The finger joints are referred to as the DIP, PIP and MCP joints in the following. These terms are short for “distal interphalangeal”, “proximal interphalangeal”, and “metacarpophalangeal”, respectively. Some parts of the exo-tendons 5 are inserted through a Bowden cable 7 as will be described in further details below. The power transmission 4 comprises an anti-derailment mechanism which will be described in details in relation to FIG. 14.

The relation between the DIP and PIP joint angle trajectories for voluntary finger flexion is shown in FIG. 1.b. By “voluntary” is meant the natural movement of the hand without the hand orthosis. This relation is compared to the relation between the trajectories of the same angles while performing finger flexion using the device, i.e. the hand orthosis according to the invention. This is marked as “soft exoskeleton” in the figure. The straight line shows the voluntary movement, and the region above this straight line represents the comfortable region where the applied movements feel natural to the user. The region below the straight line represents the uncomfortable region. The most comfortable experience to the user will be as close to the straight line as possible. The figure shows that the relation between the DIP and PIP joint angles, generated by flexing the finger using the device, is always close to the relation for voluntary finger flexion.

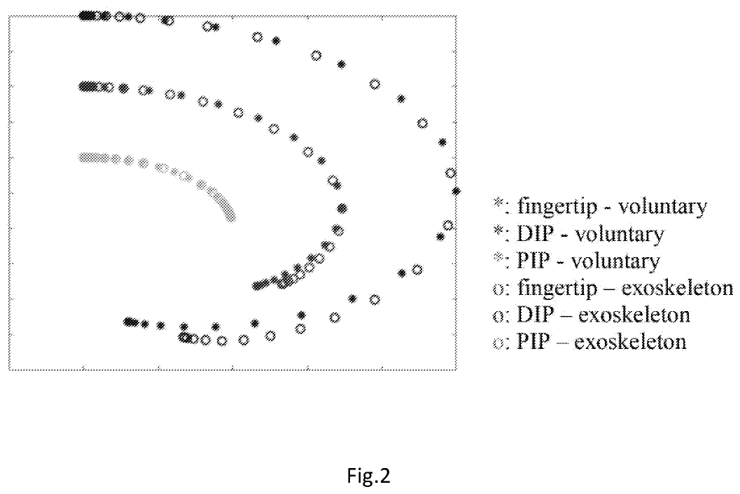

The flexion profile of the finger joints for both voluntary flexion and soft exoskeleton movement are shown in FIG. 2. Soft exoskeleton means a hand orthosis according to the invention. The angles of the finger joints were measured using a costume-made kinematic measurement glove. The measurement glove depends on flexible bend sensors (Spectrasymbol inc., 5.5 cm length) attached directly over the glove and on top of the finger joints. At the joints, two sensors are attached opposite to each other to measure the bending angles in both directions. To collect the data shown in this figure, the subjects were asked to wear the hand orthosis and on top of it, they wore the kinematic measurement glove. The subject started the experiment by voluntarily flexing the fingers from the full extension position to full flexion position. Then, data was collected again but this time, the fingers were flexed by the aid of the orthosis.

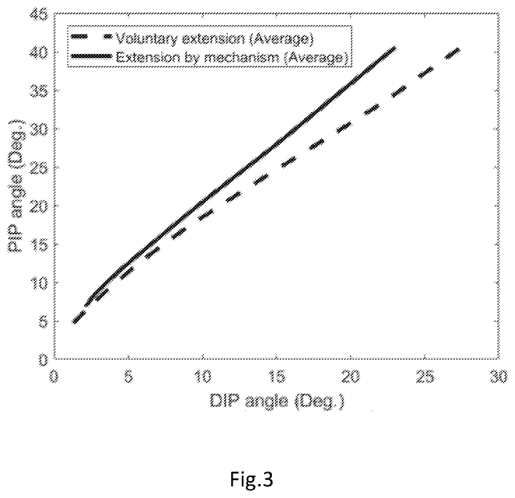

FIG. 3 represents the relation between the DIP and PIP joint angle trajectories for voluntary finger extension. This relation is compared to the relation between the trajectories of the same angles that were generated when performing finger extension using the device. The x-axis represents the DIP joint angle and the Y-axis represents the PIP joint angle. The figure shows that the relation between the DIP and PIP joint angle, generated by extending the finger using the device, is always close to the relation for voluntary finger extension. The costume-made kinematic glove, described earlier, was used to collect the data for this curve. To collect the data shown in this figure, the subjects were asked to wear the orthosis and on top of it, they wore the kinematic measurement glove. The subject started the experiment by voluntarily extending the fingers from the fingers neutral position to the full-extended position. Then, data was collected again but this time, the fingers were opened by the aid of the orthosis.

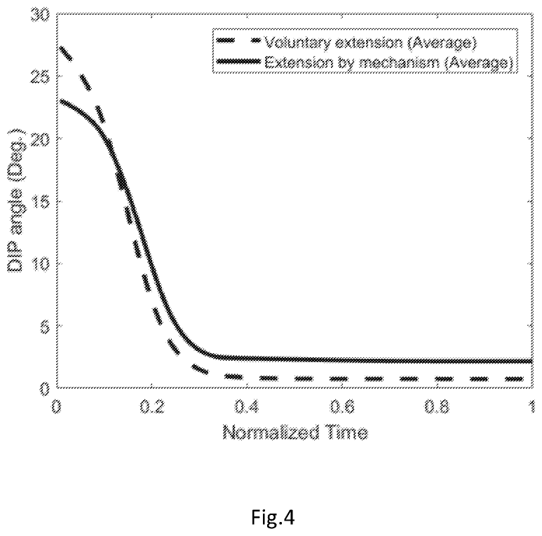

FIG. 4 represents DIP joint angle trajectory for voluntary finger extension. It is compared to the same angle trajectory that was generated when performing finger extension using the device. The Y-axis represents the DIP joint angle during the extension process. The figure shows that the orthosis could extend the fingers without hyperextending the distal phalanx, as the DIP angle is always positive as shown on the y-axis. The data was collected using the same method used for FIG. 3.

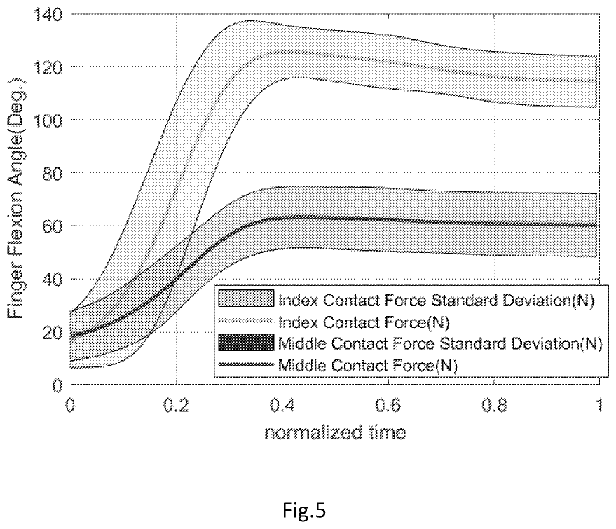

FIG. 5 represents the index and middle fingers when the middle finger is stopped, during finger flexion, to test the ability of the device to perform different grasping types. By “stopped” is meant that the mini-low power solenoid blocks the exo-tendon, that is responsible for flexing the middle finger. This does not allow the middle finger to flex any further while the other fingers are still allowed to flex. The result of the measurements relating to FIG. 5 show that it is possible to use a hand orthosis according to the invention to move one finger at a time. FIG. 5 represents the finger flexion angles of both index and middle fingers. The finger flexion angle is the sum of the three joints angles of the finger (DIP, PIP and MCP). The figure shows the average and standard deviation of the flexion angles for ten trials. Both index and middle fingers were flexed while the middle finger was stopped at angle 61°, while the index finger was left to flex to 113°.

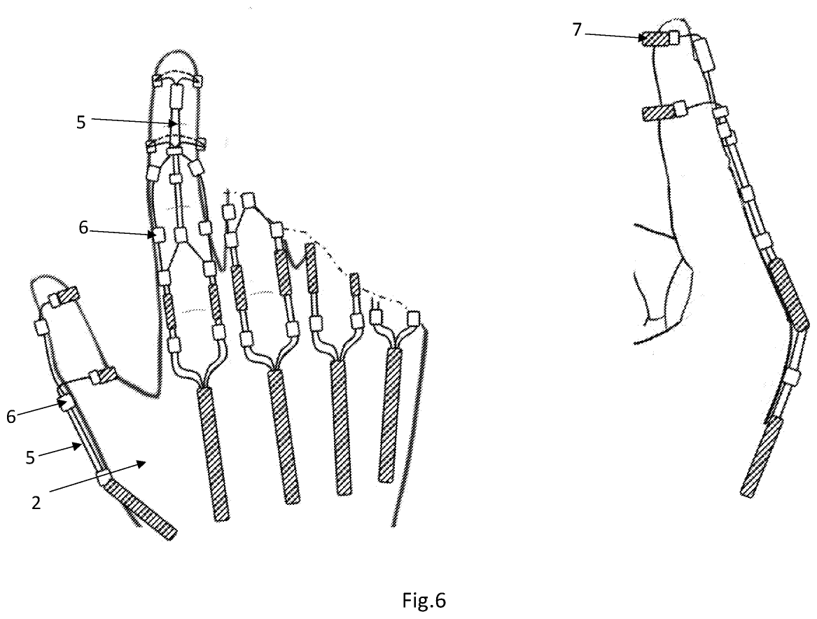

FIG. 6 schematically shows a first embodiment of the invention which can be used to replicate physiologically appropriate finger extension without suffering from hyperextension of the distal phalanx. The exo-tendons on the dorsal side of the fingers are connected to the glove, inserted to the phalanges and routed as following:
Ten guidance pearls 6 are attached to the glove 2 on the radial and ulnar sides of the proximal, middle and distal phalanges, allowing the exo-tendons 5 to pass along the radial and ulnar sides of the fingers. Four more guidance pearls 6 are located on the dorsal side of the distal, middle and proximal phalanges.
Two exo-tendons 5 are used to extend each finger. The first exo-tendon 5 passes through the guidance pearls 6 on the radial and ulnar sides of the finger, from both sides, until the middle phalanx where the exo-tendon 5 passes through the guidance pearls 6 on the dorsal side of the middle and distal phalanges. Then, it is inserted on the distal phalanx by routing it around the phalanx, using a Bowden cable 7 to prevent overtightening the tendons around it.
The other exo-tendon 5 passes through the guidance pearls 6 on the radial and ulnar sides of the finger, from both sides, until the proximal aspect of the proximal phalanx, where the exo-tendon 5 passes through the guidance pearls 6 on the dorsal side of the proximal and middle phalanges. Then, it is inserted on the middle phalanx by routing it around the phalanx, using a Bowden cable 7 to prevent overtightening the exo-tendons 5 around it.
All exo-tendons 5 pass through two pieces of Bowden cables 7 on both sides of the metacarpal joint of the finger section.

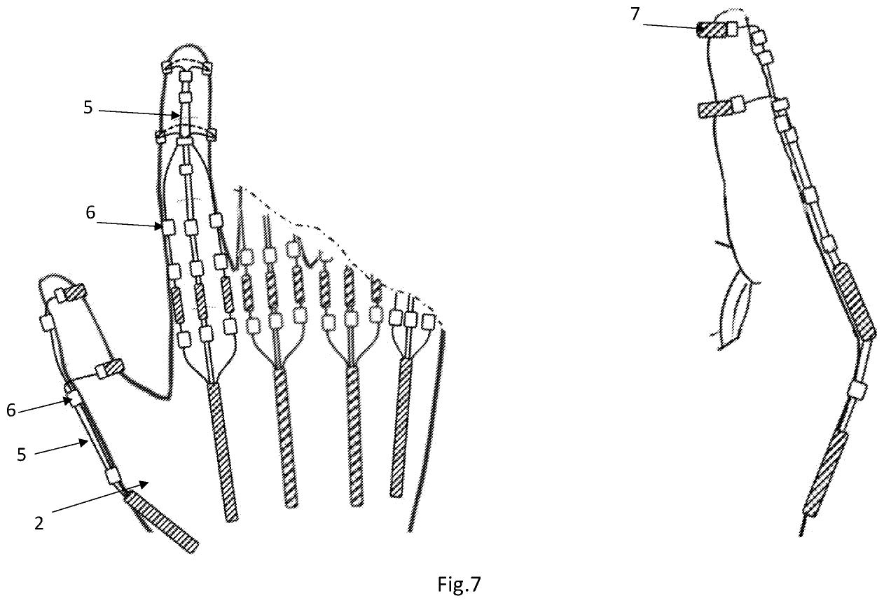

FIG. 7 schematically shows a second embodiment of the invention which can be used to replicate physiologically appropriate finger extension without hyperextension of the distal phalanx. The exo-tendons 5 on the dorsal side of the fingers are connected to the glove 2, inserted to the phalanges and routed as following:
Eight guidance pearls 6 are attached to the glove 2 on the radial and ulnar sides of the distal, proximal and middle phalanges, allowing the exo-tendons 5 to pass along the radial and ulnar sides of the finger. Six more guidance pearls 6 are located on the dorsal side of the distal, middle and proximal phalanges.
Two exo-tendons 5 are used to extend each finger. The first exo-tendon 5 passes through a separate piece of Bowden cable 7 on the radial and ulnar side of the metacarpal joint, then through the guidance pearls 6 on the radial and ulnar side of the finger, until the distal aspect of the proximal phalanx, where the exo-tendon 5 passes through the guidance pearls 6 on the distal aspect of the middle and distal phalanges on the dorsal side of the finger;
Then the first exo-tendon 5 is fixed on the distal phalanx by routing it around the phalanx;
the second exo-tendon 5 passes through a separated piece of Bowden cable 7 on the dorsal side of the metacarpal joint, then through the guidance pearls 6 on the dorsal side of the proximal phalanx, until the distal aspect of the middle phalanx;
then the second exo-tendon 5 is fixed on the middle phalanx by routing it around the phalanx.

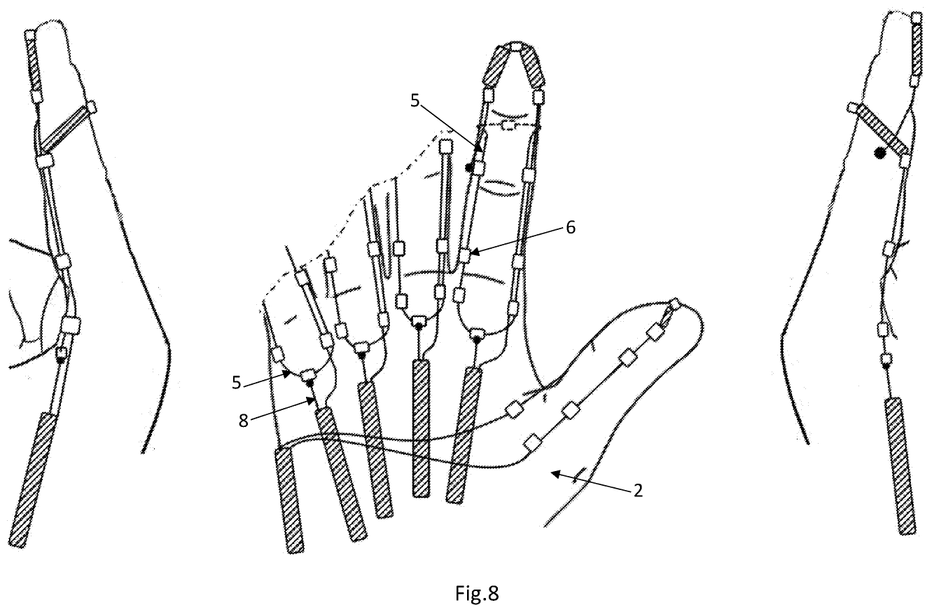

FIG. 8 schematically shows a third embodiment of the invention which can be used to replicate physiologically appropriate finger flexion. The exo-tendons 5 on the palmar side of the fingers are connected to the glove 2, inserted to the phalanges and routed as following:
Six guidance pearls 6 are attached to the glove 2 on the radial and ulnar sides of the proximal, middle, and the distal phalanges allowing the exo-tendons 5 to pass along the radial and ulnar sides of the finger. Two more guidance pearls 6 are located on the tip of the distal phalanx and on the dorsal side of the middle phalanx.
One exo-tendon 5 passes through these guidance pearls 6. The exo-tendon 5 has a starting point at the end of the first metacarpal bone before reaching the MCP joint. One end of the exo-tendon 5 passes through the guidance pearls 6 on the ulnar side of the finger, ascending upwards until the middle phalanx, and then descending on the radial side of the finger to be fixed further down beyond the starting point.
The other end of the exo-tendon 5 passes through the guidance pearls 6 on the radial side of the finger, ascending until the distal part of the distal phalanx to descend through the guidance pearls 6 on the ulnar side of the finger to be fixed at the proximal aspect of the middle phalanx.
On the distal phalanx, the exo-tendon 5 passes through a Bowden cable 7 to prevent it from being overtightened around the distal phalanx when the exo-tendons 5 are tightened.
A driver exo-tendon 8 is connected to the first exo-tendon 5 at the starting point to be pulled later using the motor in the actuator box 3.

This flexion mechanism distributes the flexion forces equally over the distal and proximal phalanges throughout the flexion motion (until full flexion), thereby mimicking the human musculoskeletal flexion system. This allows the mechanism to respect the biomechanical constraints between the distal interphalangeal joints (DIP) and the proximal interphalangeal joints (PIP) due the musculoskeletal and tendon structure, where the PIP angle should be always >1.5 times the DIP angle to have a comfortable flexion.

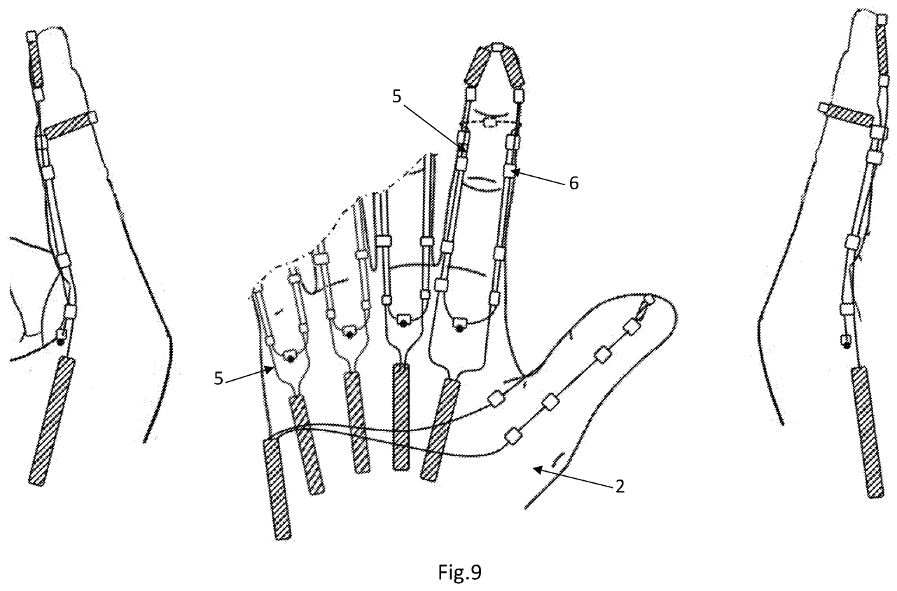

FIG. 9 schematically shows a fourth embodiment of the invention which can be used to replicate physiologically appropriate finger flexion. The exo-tendons 5 on the palmar side of the fingers are connected to the glove 2, inserted to the phalanges and routed as following:
Eight guidance pearls 6 are attached to the glove 2 on the radial and ulnar sides of the proximal, middle, and the distal phalanges allowing the exo-tendons 5 to pass along the radial and ulnar sides of the finger. Two more guidance pearls 6 are located on the tip of the distal phalanx and on the dorsal side of the middle phalanx.
An exo-tendon 5 passes through these guidance pearls 6. The exo-tendon 5 has a starting point at the end of the first metacarpal bone before reaching the metacarpophalangeal joint. One end of the exo-tendon 5 passes through the guidance pearls 6 on the ulnar side of the finger, ascending upwards until the middle phalanx, surrounding the distal part of the phalanx on the dorsal side and then descending on the radial side of the finger.
The other end of the exo-tendon 5 passes through the guidance pearls 6 on the radial side of the finger, ascending until the distal part of the distal phalanx to descend through the guidance pearls 6 on the ulnar side of the finger; and
Both ends of the exo-tendons 5 are pulled directly by the actuator box 3 to flex the finger section.
On the distal and middle phalanges, the exo-tendon 5 passes through a Bowden cable 7 to prevent it from being overtightened around the distal phalanx when the exo-tendons are tightened.

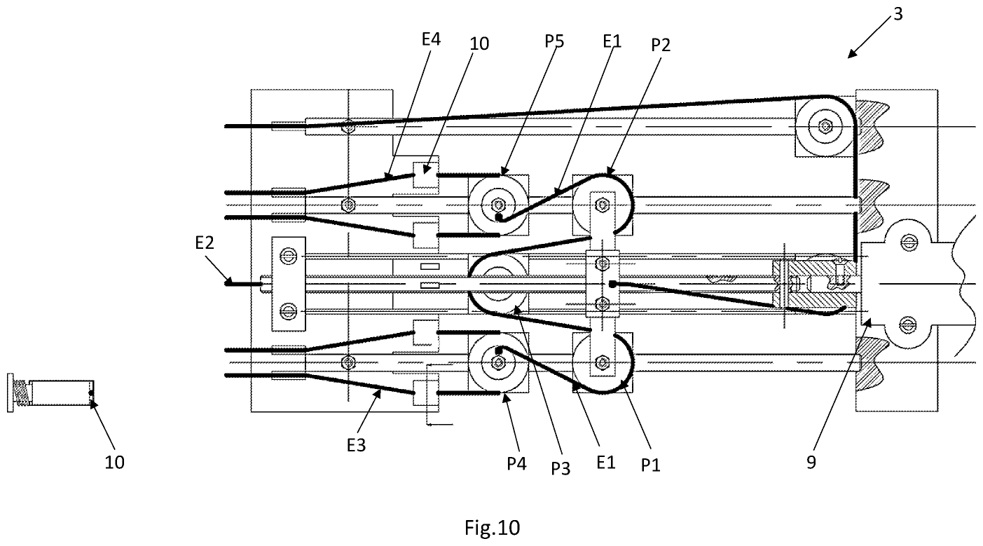

FIGS. 10-13 schematically show four different designs of the actuator box 3 that fulfil the previously mentioned features. The first is based on pulleys as shown in FIG. 10. The pulleys are divided into two levels. Three pulleys are used in the first level, as shown in FIG. 10, where pulleys P1 and P2 are pulled together by the motor 9 and pulley P3 is pulled under the effect of the tension force that is generated in exo-tendon E1, which passes around the three pulleys. Pulley P3 is connected directly with the thumb by exo-tendon E2. The two ends of exo-tendon E1 are used to connect the pulleys in the first level with the pulleys in the second level.

This mechanism divides the force applied by the motor F as follows:
F/2 for exo-tendon E2
F/4 for each of the two ends of the exo-tendon E1

Each of the F/4 forces that are applied on the two ends of exo-tendon E1 will be divided again with the pulleys in the second level. In the second level, two pulleys are used to divide the two F/4 forces that are received from the first level. Each pulley is connected to one end of exo-tendon E1. Exo-tendons E3 and E4 are routed around pulleys P4 and P5, respectively. The two ends of exo-tendon E3are connected to the index and middle fingers, while the two ends of exo-tendon E4 are connected to the ring and little fingers. Five low power solenoids are used as stoppers 10 at the end of exo-tendons E2, E3 and E4 as shown in FIG. 8. This allows the system to control each finger independently and perform different grasping types.

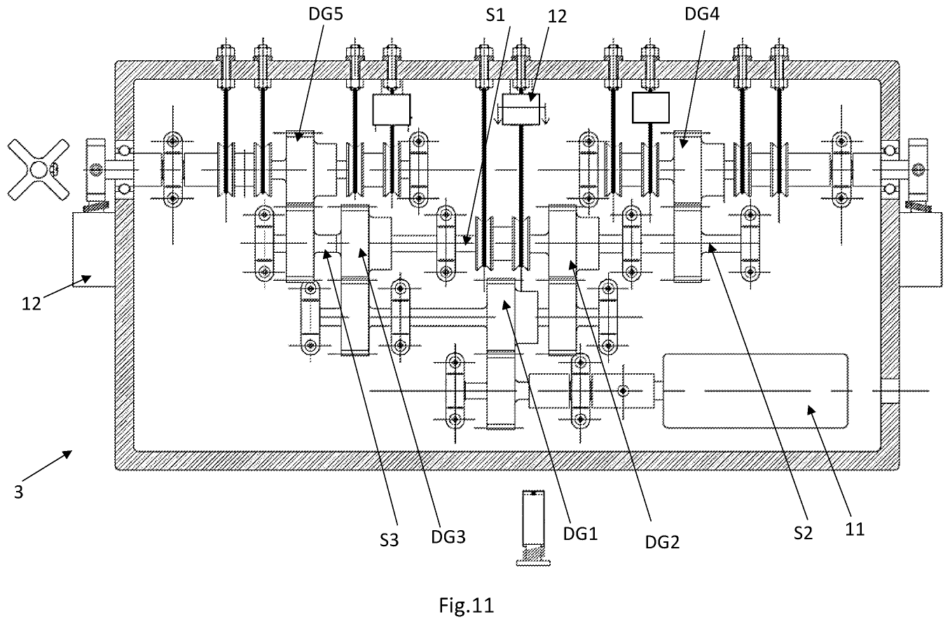

The second design of the actuator box 3 is based on differential gears as shown in FIG. 11. One DC brushless motor 11 is used to drive the differential gear set DG1. This gear set distributes the force F generated from the motor 11 equally over the differential gear sets DG2 and DG3 (which also divide the forces by two). The forces are finally distributed as following:
F/2 on shaft S1
F/4 on shaft S2
F/4 on shaft S3

Shaft S1 drives the thumb directly by connecting an exo-tendon 5 between the shaft and the thumb. Shaft S2 drives the index and the middle fingers after dividing its F/4 force equally using the differential gear set DG4. Shaft S3 also drives the ring and the little finger as shaft S2 using differential gear set DGS. Five low power solenoids 12 are used as stoppers for the exo-tendons 5. This allows the system to control fingers independently and perform different grasping types.

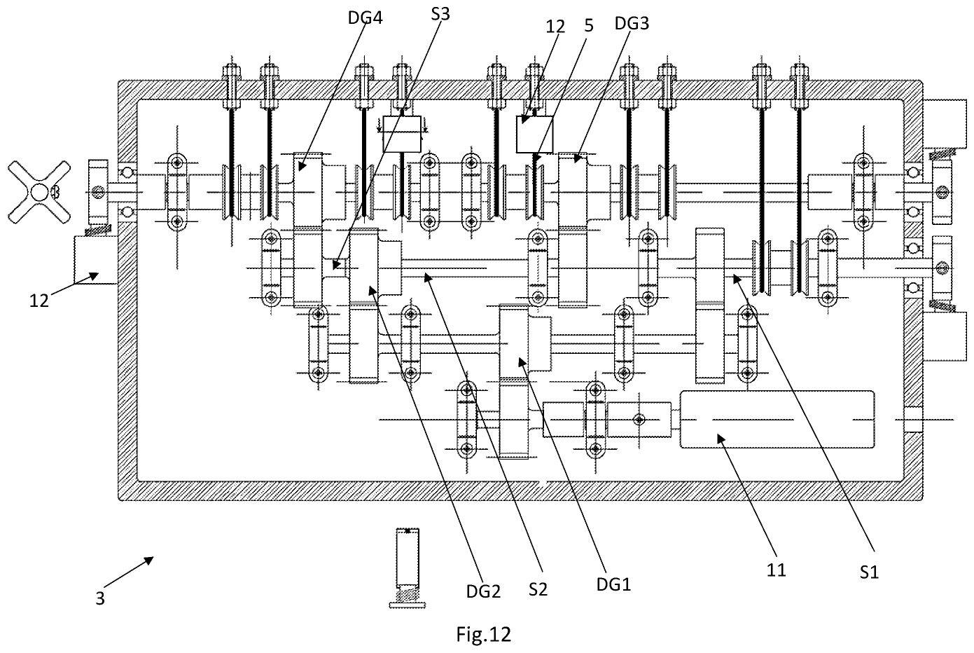

The third design of the actuator box 3 is based on differential gears as shown in FIG. 12. One DC brushless motor 11 is used to drive the differential gear set DG1. This gear set distributes the force F generated from the motor 11 equally over the differential gear set DG2 and spur gear set (which also divide the forces by two). The forces are finally distributed as following:
F/2 on shaft S1
F/4 on shaft S2
F/4 on shaft S3

Shaft S1 drives the thumb directly by connecting an exo-tendon 5 between the shaft and the thumb. Shaft S2 drives the index and the middle fingers after dividing its F/4 force equally using the differential gear set DG3. Shaft S3 also drives the ring and the little finger as shaft S2 using differential gear set DG4. Five low power solenoids 12 are used as stoppers for the exo-tendons 5. This allows the system to control fingers independently and perform different grasping types.

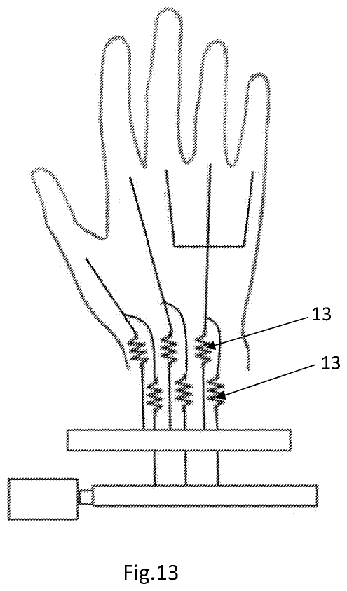

The fourth design of the actuator box 3 use springs 13 to perform different types of grasping as shown in FIG. 13. The same pulley mechanism as described in the first design, FIG. 10, is used to flex the fingers, from the palmar side. Besides using low power solenoids to perform different types of grasping, two sets of springs 13 connected to the fingers from the dorsal side could be used, as shown in FIG. 13. When the pulley system on the palmar side pulls the fingers to flex, each finger is pulled with different rate due to the springs on the dorsal side that have different spring constants. Moreover, each of the spring sets has different equilibrium position that could be changed to control the performed grasping type. The spring constants are selected using the first two principal components of “The fingers flexion angle vector” (i.e. it is a vector that represents the flexion angles of the fingers for each grasping type).

FIG. 14 shows the anti-derailment pulley mechanism. It consists mainly from two blocks 14. A cable passes through the two blocks 14 and meanwhile it is routed around two pulleys P4,P5 opposite to each other. The cable 15 has two knots 16 located directly behind the blocks 14. The knots 16 are located in a way that oblige the blocks 14 to move opposite to each other. This mechanism is always located between the glove 2 and the actuator box 3. Therefore, the exo-tendons 5 pass through this mechanism where the flexion exo-tendon 5F passes from one side and the extension exo-tendon 5E passes from the other side for each finger section. By the aid of the other knots 17, the block of the unactuated side will always move to the opposite direction, pulling the unactuated exo-tendon, to prevent the exo-tendon to be derailed from the shaft at the actuator box 3.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Furthermore, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1. A hand orthosis for assisting the movements of a user's hand, the hand orthosis comprising:
a soft glove adapted to receive at least a part of the user's hand with finger sections of the glove receiving each of the user's fingers,
at least one actuator box, and
a power transmission for transferring forces from the at least one actuator box to different parts of the glove via exo-tendons,
wherein the exo-tendons are arranged on a dorsal side, on a palmar side, or on both a dorsal side and a palmar side of the glove, and
wherein the exo-tendons are arranged so that the glove can assist extension, flexion, or both extension and flexion of the fingers, respectively, and so that the exo-tendons act on the fingers to produce motion that mimics the movement of a human hand.

2. The hand orthosis according to claim 1, wherein the at least one actuator box comprises at least one motor for providing power to the power transmission.

3. The hand orthosis according to claim 2, wherein the at least one actuator box comprises exactly one motor.

4. The hand orthosis according to claim 1, wherein the at least one actuator box comprises at least one mini-low power solenoid to control a finger section individually by blocking/unblocking its movement.

5. The hand orthosis according to claim 1, wherein the exo-tendons are arranged and controlled so that the forces from the at least one actuator box are distributed equally between a thumb on one side and the rest of the fingers on the other side.

6. The hand orthosis according to claim 1, wherein the exo-tendons are arranged and controlled so that the finger sections of the glove can be individually activated.

7. The hand orthosis according to claim 2, wherein the exo-tendons are arranged and controlled so that motor torque is distributed equally between the thumb on one side and the rest of the fingers on the other side to provide the user with a natural grasp.

8. The hand orthosis according to claim 1, wherein the exo-tendons are attached to the glove at the radial and ulnar side of the proximal, middle and distal phalanges of each finger section of the glove.

9. The hand orthosis according to claim 1, wherein at least one of the exo-tendons passes through a Bowden cable along at least a part of its length.

10. The hand orthosis according to claim 9, wherein the exo-tendons pass through Bowden cables from the at least one actuator box to a region of the glove adapted to be located adjacent to the metacarpal bones of the user during use of the hand orthosis.

11. The hand orthosis according to claim 1, wherein the exo-tendons are fastened to the glove by use of guidance pearls attached to an outer surface of the glove.

12. The hand orthosis according to claim 11, wherein each of the exo-tendons is routed as follows around the corresponding finger section to provide a force system over the finger section that resembles the force system provided by human flexion tendons:
the exo-tendon has a starting point at the end of a first metacarpal bone before reaching a metacarpophalangeal joint, one end of the exo-tendon passes through the guidance pearls on the ulnar side of the finger, ascending upwards until a middle phalanx, surrounding the distal part of the phalanx on the dorsal side, and then descending on the radial side of the finger to be fixed further down beyond the starting point;
the other end of the exo-tendon passes through the guidance pearls on the radial side of the finger, ascending until a distal part of a distal phalanx to descend through the guidance pearls on the ulnar side of the finger, to be fixed at a proximal aspect of a middle phalanx; and
a driver exo-tendon is connected to the exo-tendon at the starting point to be pulled using the at least one actuator box.

13. The hand orthosis according to claim 11, wherein each of the exo-tendons is routed as follows to provide the force system for the corresponding finger section that resembles the force system provided by human flexion tendons:
the exo-tendon has a starting point at the end of a first metacarpal bone before reaching a metacarpophalangeal joint, one end of the exo-tendon passes through the guidance pearls on the ulnar side of the finger, ascending upwards until a middle phalanx, surrounding a distal part of the phalanx on the dorsal side, and then descending on the radial side of the finger;
the other end of the exo-tendon passes through the guidance pearls on the radial side of the finger, ascending until the a distal part of the a distal phalanx to descend through the guidance pearls on the ulnar side of the finger; and
both ends of the exo-tendons are pulled directly by the at least one actuator box to flex the finger section.

14. The hand orthosis according to claim 11, wherein the exo-tendons on the dorsal side of the finger sections of the glove are arranged so that two exo-tendons are used to extend each finger; the two exo-tendons are arranged so that:
the first exo-tendon passes through a first piece of Bowden cable on the radial and ulnar side of a metacarpal joint, then through the guidance pearls on the radial and ulnar side of the finger, until a middle phalanx, where the exo-tendon passes through the guidance pearls on the dorsal side of middle and distal phalanges;
then the first exo-tendon is fixed on the distal phalanx by routing it around the phalanx;
the second exo-tendon passes through a second piece of Bowden cable on the radial and ulnar side of the metacarpal joint, then through the guidance pearls on the radial and ulnar side of the finger, until a proximal aspect of a proximal phalanx, where the second exo-tendon passes through the guidance pearls on the dorsal side of the proximal and middle phalanges;
then the second exo-tendon is fixed on the middle phalanx by routing it around the phalanx.

15. The hand orthosis according to claim 11, wherein the exo-tendons are routed as follows to extend the corresponding finger section, using two exo-tendons:
the first exo-tendon passes through a first piece of Bowden cable on the radial and ulnar side of a metacarpal joint, then through the guidance pearls on the radial and ulnar side of the finger, until a distal aspect of a proximal phalanx, where the exo-tendon passes through the guidance pearls on the distal aspect of the middle and distal phalanges on the dorsal side of the finger;
then the first exo-tendon is fixed on the distal phalanx by routing it around the phalanx;
the second exo-tendon passes through a second piece of Bowden cable on the dorsal side of the metacarpal joint, then through the guidance pearls on the dorsal side of a proximal phalanx, until the distal aspect of the middle phalanx;
then the second exo-tendon is fixed on the middle phalanx by routing it around the phalanx.

16. The hand orthosis according to claim 1, wherein the at least one actuator box comprises at least one motor and a plurality of pulleys used to guide the exo-tendons, wherein the pulleys are adapted and arranged to enable a self-adapted distribution of the forces from the at least one motor to each of the finger sections of the glove via the power transmission.

17. The hand orthosis according to claim 1, wherein the at least one actuator box comprises at least one motor and a plurality of differential gears for providing a self-adapted distribution of the forces from the at least one motor to each of the finger sections of the glove via the power transmission.

18. The hand orthosis according to claim 1, wherein the at least one actuator box comprises at least one motor, a plurality of springs and pulleys adapted and arranged to provide a self-adapted distribution of the forces from the at least one motor to each of finger sections of the glove via the power transmission.

19. A method of using the hand orthosis according to claim 1 for assisting a task oriented rehabilitation program or to provide permanent assistance during daily life activities to chronically impaired patients or as a tool for providing virtual force resistance in virtual-reality applications.

20-21. (canceled)

22. A method of using the hand orthosis according to claim 1 to provide workers who work with heavy equipment with powerful hand grasping of objects.
